(19) United States
(12) Patent Application Publication
(10) Pub. No.: US 2025/0298038 A1
(43) Pub. Date: Sep. 25, 2025

(54) Use of reagent for detecting C1QBP protein expression level in preparation for oral cancer screening or prognosis kit

(71) Applicant: SICHUAN UNIVERSITY, Chengdu, Sichuan (CN)

(72) Inventors: Jing LI, Chengdu, Sichuan (CN); Jiongke WANG, Chengdu, Sichuan (CN); Yujie SHI, Chengdu, Sichuan (CN); Yu ZHOU, Chengdu, Sichuan (CN); Xin ZENG, Chengdu, Sichuan (CN); Qianming CHEN, Chengdu, Sichuan (CN)

(21) Appl. No.: 18/862,317

(22) PCT Filed: May 6, 2023
(86) PCT No.: PCT/CN2023/092512

§ 371 Date: Nov. 1, 2024

(30) Foreign Application Priority Data
May 9, 2022 (CN) 202210499870.2

(51) Int. Cl. G01N 33/68; G01N 33/574

(52) U.S. Cl. CPC G01N 33/6893; G01N 33/57407; G01N 33/57484; G01N 2333/4716; G01N 2800/52

(57) ABSTRACT

A C1QBP protein can be used in preparation for an oral cancer screening or prognosis marker. A reagent for detecting a C1QBP protein expression level is in preparation for an oral cancer screening or prognosis kit. The C1QBP protein expression level in an oral mucosa tissue of a patient suffering from oral squamous cell carcinoma is found to be remarkably higher than that of a healthy patient. Using a reagent for detecting a C1QBP protein expression level for preparing a screening or prognosis kit of oral squamous cell carcinoma can realize effective screening or prognosis of oral squamous cell carcinoma.

### FIELD OF THE INVENTION

The present invention relates to the field of in vitro diagnostic reagents (IVD), and specifically to the use of a reagent for detecting the expression level of C1QBP protein in the manufacture of screening or prognostic kit for oral cancer.

### BACKGROUND OF THE INVENTION

Oral squamous cell carcinoma (OSCC) is the most common oral malignancy in clinical practice. Despite the rapid development of imaging, surgery, radiotherapy, and systemic treatment techniques, the overall survival rate of OSCC patients has not been significantly improved. One of the main reasons for poor treatment efficacy is lymph node metastasis, and the mechanism for the biological behavior of OSCC malignant metastasis is not yet clear. It has been shown that there are complex metabolic changes in tumor cells, and the metabolic heterogeneity that exists during the occurrence and development of tumors is known as tumor metabolic reprogramming. Tumor metabolic reprogramming is one of the important markers for malignant tumors, and closely related to malignant behaviors such as tumor growth, invasion, metastasis, and immune escape. Tumor therapy of targeting metabolism has great clinical potential.

C1QBP, also known as gClqR, p32, p33, and HABP1, is distributed in cells and on the cell surface, and can be secreted outside cells as a secreted protein. In cells, C1QBP is mainly located in mitochondria, where its important function is to maintain oxidative phosphorylation, provide energy to cells, and keep metabolic balance. C1QBP is highly expressed in mitochondria of cells that require energy metabolism (such as the heart, skeletal muscle, testes, ovaries, small intestine, and colon). Currently, it has been shown that the expression of C1QBP is increased in many malignant tumors, such as breast cancer, lung cancer, etc. In the meanwhile, it has also been shown that C1QBP is low expressed in cervical cancer, indicating that C1QBP has a tissue-specific regulation on tumor biological behavior, but the research on the expression and function of C1QBP in OSCC has not been reported.

### CONTENTS OF THE INVENTION

The object of the present invention is to provide a novel protein marker for oral cancer, and the use of a detection reagent from the marker in the manufacture of screening or prognostic kits for oral cancer.

The technical solution of the present invention includes: The use of C1QBP protein in the manufacture of screening or prognostic markers for oral cancer.

The use of the reagents for detecting the expression level of C1QBP protein in the manufacture of screening or prognostic markers for oral cancer.

Further, the reagent for detecting the expression level of C1QBP protein is an enzyme-linked immunosorbent assay (ELISA) reagent.

Further, the reagent for detecting the expression level of C1QBP protein is an immunohistochemical (IHC) staining reagent.

Further, said oral cancer is OSCC.

More further, the reagent for detecting the expression level of C1QBP protein is a reagent for detecting the expression level of C1QBP protein in human oral mucosal tissue.

The present invention also provides a kit for screening or prognosis of oral cancer, which comprises a reagent for detecting the expression level of C1QBP protein.

Further, the reagent for detecting the expression level of C1QBP protein is an ELISA reagent.

Further, the reagent for detecting the expression level of C1QBP protein is an IHC staining reagent.

Further, said oral cancer is OSCC.

More further, the reagent for detecting the expression level of C1QBP protein is a reagent for detecting the expression level of C1QBP protein in human oral mucosal tissue.

In one important aspect, the present invention has evidenced that the expression level of C1QBP in human oral mucosal tissue is significantly correlated with the risk of developing OSCC. Therefore, the risk of OSCC can be determined by detecting the expression level of C1QBP in human oral mucosal tissue. As for the specific means of detecting the expression level of C1QBP in human blood, various available techniques in the prior art can be used. In the example of the present invention, IHC staining assay is specifically used to detect, but not limited to it. Any method that can detect the expression level of C1QBP can be used for screening or prognosis of OSCC.

The present invention provides a novel screening marker and a novel screening kit for OSCC, which can effectively screen and predict the course and outcome of OSCC, and thus have good application prospects.

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. The scoring criteria for the intensity of immunohistochemical staining.

FIG. 2. The protein expression pattern of C1QBP in various stages of oral mucosal carcinogenesis (A. The expression of C1QBP in normal oral mucosa, oral potential malignant diseases (OPMD), and oral squamous cell carcinoma (OSCC); B. The changes in the expression of C1QBP with the progression of oral mucosal carcinogenesis).

FIG. 3. The relationship between the expression of C1QBP and the prognosis of OSCC patients.

### EXAMPLES

### Example 1 the Relationship Between the Expression Level of C1QBP in Oral Mucosal Tissue and OSCC

### I. Method

### 1. Sample Collection

Firstly, normal oral mucosal tissues, lesion tissues from patients with oral potential malignant diseases (OPMD), and lesion tissues from OSCC patients were collected, including 10 cases in normal control group, 13 cases in OPMD group, and 45 cases in OSCC group. The obtained tissues were fixed, dehydrated, and embedded according to the following methods, and then paraffin sections were made.

(1) The collected tissues were immediately washed twice with PBS.

(2) The tissues were gently dried with paper and placed in a 4% paraformaldehyde solution. The volume ratio of the tissue to 4% paraformaldehyde solution was 1:30.

(3) According to the grouping, the fixed tissues were transferred into labeled embedding frames one day in advance and rinsed overnight under fine tap water.

(4) The embedding frames were placed in a dehydrator and dehydrated for 24 h.

(5) The tissue was collected from the embedding frame, soaked in wax I, II, and III, and embedded with embedding wax to obtain paraffin blocks.

(6) The microtome was set to a thickness of 5 μm and used to cut tissue paraffin sections, which were smoothed flat, put in glass slides, and then picked up, followed by labelling the groups.

(7) The glass slides were kept in a 55° C. oven for 4 h, dried, and then stored in a refrigerator at 4° C. for later use.

### 2. Sample Processing

The IHC staining of obtained tissue sections were carried out using the following method.

(1) The paraffin sections were kept in a 70° C. oven for 1 h.

(2) Dewaxing in xylene and rehydration in graded alcohols: The paraffin sections were allowed to sequentially dewax in xylene I for 20 min and xylene II for 10 min, respectively, and then rehydrated in gradient alcohols of 100%, 95%, 90%, 80%, and 70%, 5 min for each time.

(3) The paraffin sections were rinsed with distilled water twice, 5 min for each time.

(4) Antigen repair: A pressure cooker was added with water, and then the antigen retrieval kit was placed, which was filled with antigen retrieval solution. In this experiment, sodium citrate buffer (10 mM, pH 6.0) was used. The lid of the pot was not tightly closed, and it was heated to boiling in hotpot mode. The paraffin sections were transferred into the antigen retrieval kit, and then the lid was tightly closed, followed by heating to the appearance of steam and kept for 3 min. Gas was slowly released until no steam was sprayed out. The air valve was removed, and the kit was taken out, followed by removing the lid, cooling in cold water for 20 min, as well as rinsing with TBS twice, 5 min for each time.

(5) Treatment with 3% hydrogen peroxide: 20 mL of 30% H₂O₂ and 180 mL of distilled water were mixed, and then the paraffin sections were soaked in H₂O₂ and kept at room temperature in the dark for 20 min, followed by rinsing with TBS for three rime, 5 min for each time.

(6) The secondary antibody solution A (EnVision+System, Dako Co.) was added dropwise, and then the paraffin sections were blocked at room temperature for 30 min, followed by rinsing with TBS for three rime, 5 min for each time.

(7) The primary antibody was added dropwise according to the dilution ratio, followed by incubating at room temperature for 30 min and then incubating overnight at 4° C. in a refrigerator.

(8) After incubating overnight, the paraffin sections were taken out and rinsed with TBS for three rime, 5 min for each time.

(9) The secondary antibody solution B (EnVision+System, Dako Co.) was added dropwise, and then incubated for 60 min, followed by rinsing with TBS for three rime, 5 min for each time.

(10) DAB color developing solution (diluted in 1:50) was added dropwise to each section, and then it was observed under a microscope. When the section showed tawny, it was placed in clean water to terminate.

(11) The sections were counterstained with hematoxylin for 1 min, rinsed with distilled water, separated with 2% hydrochloric acid in alcohol for 1 second, washed with distilled water for 15 min, and then returned to blue with ammonia solution.

(12) The sections were rinsed with running clean water for 20 min.

(13) The sections were dried by gradient alcohol dehydration (successively with 75% alcohol, 80% alcohol, 90% alcohol, and 100% alcohol, 5 min for each time), and then cleared in xylene (with xylene I and xylene II in sequence, 5 min for each time).

(14) The sections were sealed with neutral gum and naturally dried.

(15) The sections were scanned, and the image data were copied to CDs.

### 3. Result Statistics

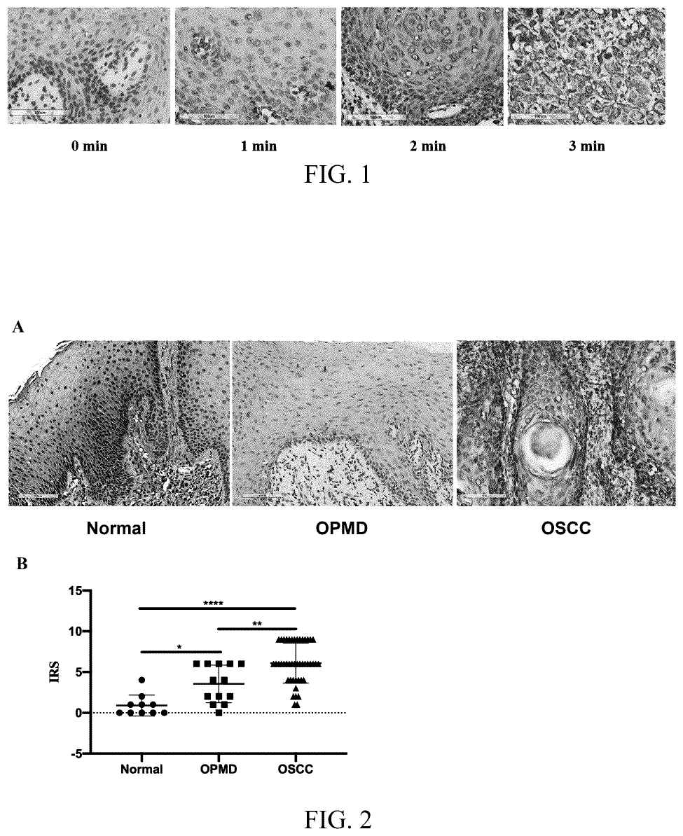

5 non-repetitive fields on each slice were observed using a high-power microscope, and C1QBP was evaluated as positive for the presence of yellow or tawny particles in the cytoplasm. The average staining intensity of the entire slice and the percentage of positive cells to tumor cells in each field were evaluated, so as to perform immunohistochemical staining scoring (IRS). All slices underwent blind testing by two experienced researchers to evaluate and calculate the average score for each slice. The scoring criteria were as follows (Table 1 and FIG. 1)

TABLE 1
IHC staining scoring criteria.

| Staining intensity (A) | Stained area (B) | Staining score (A*B) |
|---|---|---|
| 0, no staining | 0 (<5%) | 7 scores in total: |
| 1, light yellow, weakly | 1 (5%-40%) | 0, 1, 2, 3, 4, 6, 9 |
| positive | | |
| 2, medium yellow, | 2 (41%-70%) | |
| medium positive | | |
| 3. dark yellow, strongly | 3 (71%-100%) | |
| positive | | |

### 4. Result Analysis

By statistical analysis, the expression level of C1QBP in normal oral mucosal tissue was relatively low (with an average score of 0.900), while the expression level of C1QBP in damaged tissue of patients with potential malignant oral diseases (OPMD) was increased (with an average score of 3.538). The expression level of C1QBP in OSCC patient tissue was relatively strongest (with an average score of 6.067). There was a statistically significant difference between OPMD group and normal oral mucosal group (P<0.05), between OSCC group and normal oral mucosal group (P<0.001), as well as between OSCC group and OPMD group (P<0.01). The expression level of C1QBP is negatively correlated with the prognosis of OSCC patients, and the differences are statistically significant (P<0.05).

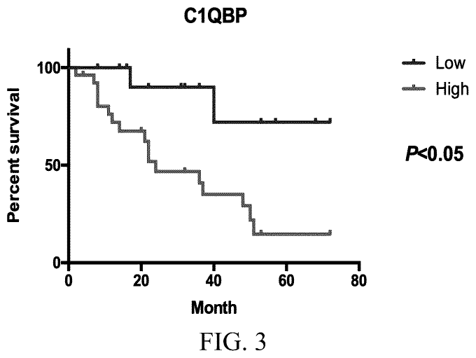

Based on the above results, it was shown that the expression level of C1QBP was increased with the progression of oral mucosal carcinogenesis (FIG. 2), and was negatively correlated with the prognosis of OSCC patients (FIG. 3). By detecting the expression level of C1QBP in oral mucosal tissue, the objective of screening OSCC or predicting the future course and outcome of OSCC could be achieved.

### Example 2 Components and Usage Method of the Detection Kit of the Present Invention

### I. Components of Kit

| | Amount |
|---|---|
| Components for preparing paraffin | |
| section | |
| PBS buffer | One bottle, 60 mL for each bottle |
| 4% Polyformaldehyde | One bottle, 30 mL for each bottle |
| wax I, wax II, wax III, embedding | One bottle for each wax, 30 mL |
| wax | for each bottle |
| Components for immunohisto- | |
| chemical staining | |
| Xylene | One bottle, 30 mL for each bottle |
| 100%, 95%, 90%, 80%, 75%, and | One bottle for each alcohol, 30 mL |
| 70% alcohol | for each bottle |
| Sodium citrate buffer (10 mM, | One bottle, 30 mL for each bottle |
| pH 6.0) | |
| 3% hydrogen peroxide | One bottle, 30 mL for each bottle |
| The secondary antibody solution A | One bottle, 1 mL for each bottle |
| (En Vision + System, Dako Co.) | |
| The secondary antibody solution B | One bottle, 1 mL for each bottle |
| (En Vision + System, Dako Co.) | |
| C1QBP primary antibody | One bottle, 1 μL for each bottle |
| DAB color developing solution | One bottle, 1 mL for each bottle |
| Hematoxylin | One bottle, 1 mL for each bottle |
| 2% Hydrochloric acid in alcohol | One bottle, 30 mL for each bottle |
| Ammonia | One bottle, 30 mL for each bottle |
| Neutral gum | One bottle, 1 mL for each bottle |
| Distilled water | One bottle, 500 mL |

### II. Instructions for Using the Kit

Same as “Preparation of paraffin section” and “IHC staining” in Example 1.

The kit of the present invention could be used to detect the expression level of C1QBP in human oral mucosal tissue and determine the risk of OSCC. If the expression level of C1QBP was low (relative to healthy individuals), the risk of developing oral squamous cell carcinoma was low; If the expression level of C1QBP was high, the risk of developing OSCC was high, and as the expression level of C1QBP increased, the prognosis became worse, that is, the course of OSCC was longer, and the outcome was not ideal. It could be used as an auxiliary diagnosis for OSCC in clinical practice, and provide effective basis for patients to take relevant treatment measures or make decisions, with good clinical application prospects

1. C1QBP protein for use in the manufacture of screening or prognostic markers for oral cancer.

2. The reagents for detecting the expression level of C1QBP protein for use in the manufacture of screening or prognostic markers for oral cancer.

3. The use according to claim 2, characterized in that the reagent for detecting the expression level of C1QBP protein is an enzyme-linked immunosorbent assay (ELISA) reagent.

4. The use according to claim 2, characterized in that the reagent for detecting the expression level of C1QBP protein is an immunohistochemical (IHC) staining reagent.

5. The use according to claim 2, characterized in that said oral cancer is oral squamous cell carcinoma (OSCC).

6. The use according to claim 2, characterized in that the reagent for detecting the expression level of C1QBP protein is a reagent for detecting the expression level of C1QBP protein in human oral mucosal tissue.

7. A kit for screening or prognosis of oral cancer, characterized in that it comprises a reagent for detecting the expression level of C1QBP protein.

8. The kit according to claim 7, characterized in that the reagent for detecting the expression level of C1QBP protein is an ELISA reagent or an IHC staining reagent.

9. The kit according to claim 7, characterized in that said oral cancer is OSCC.

10. The kit according to claim 7, characterized in that the reagent for detecting the expression level of C1QBP protein is a reagent for detecting the expression level of C1QBP protein in human oral mucosal tissue.
